(19) United States
(12) Patent Application Publication
(10) Pub. No.: US 2012/0012537 A1
(43) Pub. Date: Jan. 19, 2012

(54) Fluid Reconditioning System and Method of Using the Same

(75) Inventor: Scott DeCoster, Belleville, MI (US)

(73) Assignee: FORD GLOBAL TECHNOLOGIES, LLC, Dearborn, MI (US)

(21) Appl. No.: 13/021,911

(22) Filed: Feb. 7, 2011

Related U.S. Application Data
(60) Provisional application No. 61/364,094, filed on Jul. 14, 2010.

(51) Int. Cl. C02F 1/32; A61L 2/10; A61L 2/08

(52) U.S. Cl. 210/748.11; 210/748.01; 250/435; 250/432 R

(57) ABSTRACT

In one embodiment, a fluid recondition system includes a container for storing a fluid; a fluid reconditioning module including a microbe-reducing device; and a first and second conduits transporting fluid between the container and the fluid reconditioning module. In another embodiment, the fluid reconditioning module includes an electromagnetic energy emitting device such as a number of ultraviolet lamps.

### CROSS-REFERENCE TO RELATED APPLICATION

This application claims the benefit of U.S. provisional Application No. 61/364,094 filed Jul. 14, 2010. The disclosure of which is incorporated in its entirety by reference herein.

### BACKGROUND

1. Technical Field

One or more embodiments of the present invention relate to a fluid reconditioning system, and particularly a fluid reconditioning system for reducing microbe count in fluids using vehicle painting process.

2. Background Art

Vehicle painting processes often involve the formation of a large amount of wash fluids that may be contaminated with dirt and grease derived from vehicle surfaces. As the washing process is carried out at warm temperatures, microbe and particularly bacteria tend to accumulate by feeding on the dirt and grease in the washing fluids. As using new batches of fresh fluids is prohibitive both due to cost and environment concerns, it is desirable to recondition these wash fluids for reuse.

### SUMMARY

In one aspect, a fluid reconditioning system is provided. In one embodiment, the fluid reconditioning system includes a fluid-storing container, a microbe-reducing device, a first conduit communicating fluid from the fluid-storing container to the microbe-reducing device, and a second conduit in fluid communication with the microbe-reducing device and the fluid storing container. In another embodiment, the second conduit communicates fluid from the microbe-reducing device to the fluid-storing container, the fluid returning to the fluid-storing container from the microbe-reducing device having a lower microbe concentration than the fluid entering the microbe-reducing device from the first conduit.

In another embodiment the fluid reconditioning module includes an electromagnetic energy emitting device. In yet another embodiment, the electromagnetic energy emitting device includes an ultraviolet energy emitting device. In yet another embodiment, the ultraviolet energy emitting device includes a number of ultraviolet lamps. In yet another embodiment, the ultraviolet lamps are in direct contact with a fluid flow.

In yet another embodiment, the fluid reconditioning system further includes first and second pressure monitors in communication with the first and second conduits, respectively. In yet another embodiment, the first and second conduits are formed of stainless steel. In yet another embodiment, the fluid reconditioning system further includes a bypass conduit for conducting fluid flow away from the fluid reconditioning module. In yet another embodiment, the fluid has a flow rate of 150 to 450 gallons per minute. In yet another embodiment, the fluid of the container includes a phosphate rinse solution used in vehicle painting processes.

In another aspect, a system for reducing microbe count in a vehicle wash fluid is provided to include a container for storing a vehicle wash fluid; and a fluid reconditioning module including an ultraviolet energy emitting device and being in fluid communication with the container.

In yet another aspect, a method for reconditioning a fluid in a container is provided to include the steps of: contacting the fluid with a fluid reconditioning module to generate a reconditioned fluid, the fluid regenerating module including an electromagnetic energy emitting device; and returning the reconditioned fluid back to the container.

### BRIEF DESCRIPTION OF THE FIGURES

FIG. 1 depicts a vehicle painting process;

FIG. 2 depicts a vehicle washing sub-process of the painting process illustrated in FIG. 1;

FIG. 3 depicts a fluid reconditioning system according to one or more embodiments of the present invention;

FIG. 4 depicts a cross-section of the fluid reconditioning module of the fluid reconditioning system of FIG. 3; and

FIGS. 5-6 depict bacteria count reduction with the use of fluid reconditioning system according to one or more embodiments of the present invention.

### DETAILED DESCRIPTION

As required, detailed embodiments of the present invention are disclosed herein. However, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for the claims and/or a representative basis for teaching one skilled in the art to variously employ the present invention.

Moreover, except where otherwise expressly indicated, all numerical quantities in this description and in the claims indicating amounts of materials or conditions of reactions and/or use are to be understood as modified by the word “about” in describing the broadest scope of this invention. Practice within the numeral limit stated is generally preferred. Also, unless expressly stated to the contrary, percent, “parts of”, and ratio values are by weight and the description of a group or class of materials are suitable or preferred for a given purpose in connection with the invention implies that mixtures of any two or more members of the group or class may be equally suitable or preferred.

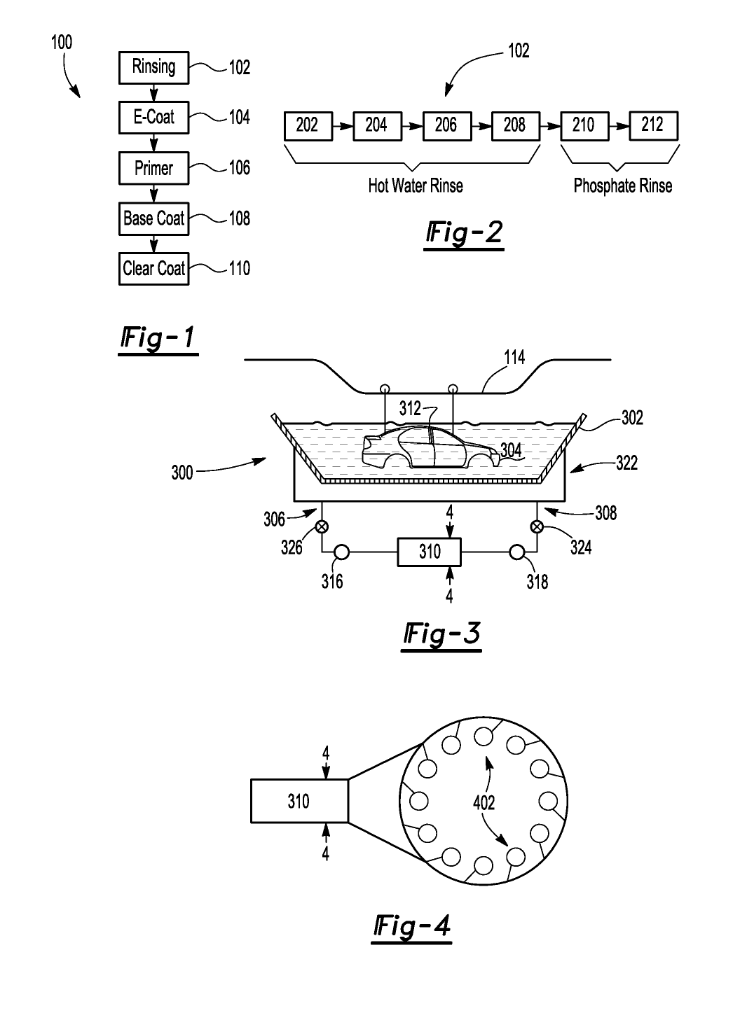

A painting process for a vehicle may be performed as illustrated in FIG. 1. At step 102, a rinsing sub-process is carried out to remove dirt and grease on the vehicle surface and ready the vehicle for subsequent painting procedures. At step 104, an E-coat sub-process is carried out to electrically dip-apply anti-corrosion layer of paint. At step 106, a primer coating sub-process is carried out to provide additional anti-corrosion protection and to conceal small surface defects. The primer coating composition can be applied in a first paint booth, typically referred to as a “primer booth.” The primer coat composition is then cured to form a primer coat over the vehicle body. The vehicle is then passed through a second paint booth, typically referred to as an “enamel booth” or a “base coat booth” where the vehicle body is then painted with a base coat composition over the cured primer coat. Application of the base coat is generally shown at step 108. At step 110, a clear coat sub-process is carried out to provide sheen to the vehicle surface. In a wet-on-wet coating process, the clear coat composition is typically applied to the vehicle body over the still wet base coat composition. The base coat and clear coat compositions are then cured together in an oven to form a base coat and a first clear coat over substantially the entire vehicle body.

Many variations may be implemented to carry out the vehicle painting process as illustrated in FIG. 1. For instance, between the E-coat sub-process 104 and the primer coat sub-process 106, the vehicle surface may be further treated with sanding, water-proof sealing, and/or anti-corrosion polymer layering.

The rinsing sub-process 102 as referenced in FIG. 1 can itself include multiple steps. By way of example, and as depicted in FIG. 2, the rinsing sub-process generally shown at 102 includes four individual hot water rinse steps 202, 204, 206, 208, and two phosphate rinse steps 210, 212. The total number of hot rinse steps and phosphate rinse steps can be any suitable number based on the particular painting project at hand. Hot water rinse helps reduce surface dirt, debris, and grease on the vehicle body; and phosphate rinse helps provide acid deposition in preparation for subsequent E-coat sub-process 104 in FIG. 1. Additional rinsing steps may be added between the sub-processes 104 and 106 to rinse off excess phosphate from the vehicle body.

Elevated temperatures are used to facilitate the rinsing. As the vehicle passes through one or more of the steps 202 to 212 illustrated in FIG. 2, microbes tend to accumulate by feeding on the dirt and grease carried over from these steps. When accumulated to a certain extent, the microbe population often results in unnecessary system clotting and associated repair costs.

To reduce microbe count and particularly bacteria count, various anti-microbe chemicals have been proposed. Examples of these chemicals include peroxide, Kathon™, and Canguard™. These chemicals have been met with limited use due to material cost and implementation complexity. By way of example, significant costs may be incurred on a weekly basis to chemically treat the vehicle rinsing fluids to an industrially acceptable level. Further, certain chemicals require particular application protocols to be fully effective and therefore introduce additional labor and equipment costs. In one or more embodiments and as will be described in more detail herein below, the present invention provides a cost effective solution to the above-identified problems.

In one or more embodiments, the term “microbe” may refer to an organism that is microscopic, or too small to be seen by the naked human eye. Non-limiting examples of microbes include bacteria, fungi, archaea, protists, microscopic plants such as green algae, microscopic animals such as plankton and planarian, or viruses.

In one embodiment, and as depicted in FIG. 3, a fluid reconditioning system generally shown at 300 is provided to include a fluid container 302 and a fluid reconditioning module 310. The fluid reconditioning module 310 is in fluid communication with the fluid container through first and second conduits 306, 308. A portion of the fluid 304 in the fluid container 302 may enter and thereafter exit the fluid reconditioning module 310 via the first and second conduits 306, 308, respectively.

In certain instances, and as depicted in FIG. 3, pressure monitors 316, 318 may be installed in the inlet 306 and the outlet 308, respectively, to monitor a pressure differential in the fluid before and after the fluid reconditioning module 310. If the pressure differential is greater than certain predetermined pressure values, it is indicative that the fluid reconditioning module 310 is operating under sub-optimal condition, for instance, unnecessarily high resistance for fluid flow has occurred, which could in turn be due to debris accumulation and clot formation. Therefore, in these instances, the pressure monitors 316, 318 collectively provide a mechanism for checking operating integrity of the fluid reconditioning module 310.

In the event when no fluid reconditioning is desired or when the fluid reconditioning module 310 is down for maintenance, the fluid 304 passes instead through the bypass conduit 322. First and second bypass valves 324, 326 may be implemented to cause flow switch between the bypass conduit 322 and the fluid reconditioning module 310.

The first and second conduits 306, 308 and the bypass conduit 322 may be formed of any suitable material, including metal and polymer and particularly stainless steel and the like.

In certain instances, the container and the fluid reconditioning module 310 may be disposed alongside each other or may be disposed on different planes with a vertical difference there between, for instance, in an upstairs-downstairs arrangement.

In certain instances, and as depicted in FIG. 3, the fluid reconditioning process may take place while a vehicle 312 is being dipped in and out of the fluid 304. The vehicle 312 may be lifted up from and lowered into the fluid 304 through a lifting bar 114.

The fluid reconditioning module 310 functions in general to reduce the microbe count, and particularly bacterial count, in the fluid 304. To do so, the fluid reconditioning module 310 is equipped with an electromagnetic energy emitting device such as an ultraviolet energy emitting device, which in certain instances may be formed of a number of ultraviolet energy emitting lamps. Without being limited to any particular theory, it is believed that the electromagnetic energy emitting device such as ultraviolet lamps emit energies that are destructive to microbe formation and/or propagation.

In certain instances, and as depicted in FIG. 4 which shows a cross-sectional view of the fluid reconditioning module 310, a number of electromagnetic energy emitting lamps such as ultraviolet lamps 402 are attached to an inner surface of the fluid reconditioning module 310. Any one of the ultraviolet lamps 402 can be configured to emit electromagnetic energy at a wavelength suitable for deactivating microbe and hence reducing microbe count. The ultraviolet lamps 402 can be of any suitable number and shape based on a particular fluid regenerating project at hand. In certain particular instances, the fluid regenerating module 310 includes 4 to 24 electromagnetic energy generating bulbs along a cross-sectional dimension and 4 to 24 along a longitudinal dimension.

The electromagnetic energy used herein, according to one or more embodiments, includes intense radio waves, microwaves, infrared waves, ultraviolet (UV) waves, X-rays, γ-rays, and any combinations thereof. In certain instances, the electromagnetic energy used herein includes UV waves with wavelength ranging from 121 nm to 10 nm for extreme UV (EUV), 150 nm to 10 nm for super UV (SUV), 100 nm to 88 nm for low UV (LUV), 200 nm to 100 nm for vacuum UV (VUV), 200 nm to 122 nm for far UV (FUV), 280 nm to 100 nm for C-wave UV (UVC), 300 nm to 200 nm for middle UV (MUV), 315 nm to 280 nm for medium UV (MUV), 400 nm to 300 nm for near UV (NUV), and 400 nm to 315 nm for UV A-wave (UVA).

The fluid reconditioning system 300, according to one or more embodiments of the present invention, provides certain cost benefits realized through the synergistic coupling of the fluid flow from and to the fluid container 302 and the microbe reducing effectuated by the fluid regenerating module 310. Without wanting to be limited to any theory, it is believed that the use of a generally long-lasting electromagnetic energy generating device for reducing microbe count in the flow through fluid substantially relieves the need for otherwise costly chemicals and complex implementation procedures for using the same. On the other hand, the relatively high flow rate generally in a range of 150 to 450 gallons per minute effectively removes along with the fluid flow any film which otherwise would be formed on the surfaces of the electromagnetic energy generating devices such as UV bulbs. Thus, active use period of the electromagnetic energy generating devices can be substantially extended.

The ultraviolet emission may be carried out using light of a wavelength in a range of between 200 nanometers to about 600 nanometers, particularly of between 250 nanometers to about 500 nanometers, and more particularly of between 300 nanometers to about 450 nanometers. In certain particular instances, the UV irradiation may be carried out using light of UVA rays with a wavelength ranging from 300 to 400 nanometers as compared to UVB rays or UVC rays. The use of UVA rays having relatively longer wavelength, when coupled with the much shortened irradiation treatment period due to the synergistic collaboration afforded by the IR exposure according to embodiments of the present invention, provides an additional benefit in reducing or eliminating ozone production otherwise conventionally associated with the use of UV light.

The UV irradiation may be carried out using any suitable UV lamps as the light source. Both point sources and platform projectors such as lamp carpet may be used. The UV light sources illustratively include carbon arc lamps, xenon arc lamps, pressurized mercury lamps, metal halide lamps, microwave-excited metal-vapor lamps, excimer lamps, UV fluorescent lamps, argon filament lamps, electronic flash lamps, and photographic flood lights. The UV light sources such as the above-mentioned pressurized (high-, medium-, or low-) mercury vapor lamps may further be doped, for instance with lead, to open up a radiation window.

The UV irradiation steps can be of any suitable dosage. Selecting a suitable dosage may depend on several parameters including, among others curing duration, fluid flow rate, fluid density, and microbe content. In at least one particular embodiment, and as measured with a wavelength of between 320 to 390 nanometers, the first dose and the second dose are each independently a value in a range of 10³ to 6×10⁴ J/m², in a range of 2×10³ to 5×10⁴ J/m², or in a range of 6×10³ to 4×10⁴ J/m².

In one or more embodiments, the term “vehicle” may be any vehicle capable of holding 2, 4, 7, or more passengers and may be a passenger bus, a cargo transporting truck, or the like.

### EXAMPLE

A fluid reconditioning module constructed according to FIG. 3 is tested for bacterial growth retardation in a vehicle phosphate rinse fluid. The fluid reconditioning module is equipped with 254 nanometers ultraviolet bulbs and is provided with an inner volume capable of delivering 320 fluid gallons per minute. 240 Volts and 50 Hertz are supplied to the fluid reconditioning module.

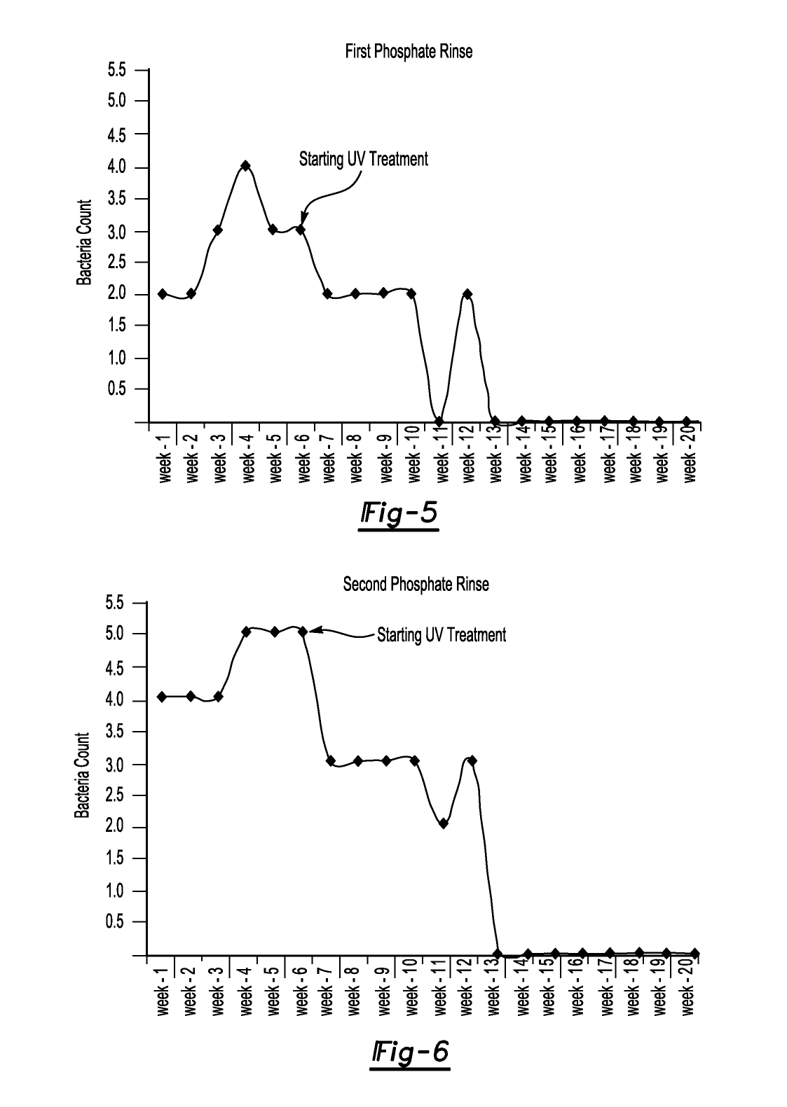

As depicted in FIGS. 5 and 6, bacteria culture colonies are found to decrease significantly on a weekly basis. Because of this reduction in bacterial count, weekly supplementation of anti-microbe chemicals is reduced to about half of the previous usage. On average, a bacteria count of 10 to zero or 10 to one can be maintained and an approximate $1,000 in weekly chemical cost savings can be realized.

While the best mode for carrying out the invention has been described in detail, those familiar with the art to which this invention relates will recognize various alternative designs and embodiments for practicing the invention as defined by the following claims.

What is claimed:

1. A fluid reconditioning system comprising:
a fluid-storing container;
a microbe-reducing device;
a first conduit communicating fluid from the fluid-storing container to the micro-reducing device; and
a second conduit in fluid communication with the microbe-reducing device and the fluid-storing container.

2. The fluid reconditioning system of claim 1, wherein the second conduit communicates fluid from the microbe-reducing device to the fluid-storing container, the fluid returning to the fluid-storing container from the micro-reducing device having a lower microbe concentration than the fluid entering the microbe-reducing device from the first conduit.

3. The fluid reconditioning system of claim 1, wherein the microbe-reducing device includes an electromagnetic energy emitting device.

4. The fluid reconditioning system of claim 3, wherein the electromagnetic energy emitting device includes an ultraviolet energy emitting device.

5. The fluid reconditioning system of claim 4, wherein the ultraviolet energy emitting device includes a number of ultraviolet lamps.

6. The fluid reconditioning system of claim 5, wherein the ultraviolet lamps are in direct contact with a fluid flow.

7. The fluid reconditioning system of claim 1, further comprising first and second pressure monitors in communication with the first and second conduits, respectively.

8. The system of claim 1, wherein the first and second conduits include stainless steel.

9. The fluid reconditioning system of claim 1, wherein the microbe-reducing device is situated below the fluid-storing container.

10. The fluid reconditioning system of claim 1, further comprising a vehicle fluid wash situated within the fluid-storing container and circulating through the first conduit, the microbe-reducing device, and the second conduit.

11. A system for reducing microbe count in a vehicle wash fluid, comprising:
a container for storing a vehicle wash fluid; and
a fluid reconditioning module including an ultraviolet energy emitting device and being in fluid communication with the container, the fluid reconditioning module reducing the microbe count of the vehicle wash fluid.

12. The system of claim 11, further comprising first and second conduits transporting the vehicle wash fluid between the container and the fluid reconditioning module.

13. The system of claim 11, wherein the ultraviolet energy emitting device includes a number of ultraviolet lamps attached to an inner surface of the fluid reconditioning module.

14. The system of claim 11, wherein the vehicle wash fluid further includes phosphate.

15. A method for reconditioning a fluid in a container, comprising:
contacting the fluid with a fluid reconditioning module to generate a reconditioned fluid, the fluid regenerating module including an electromagnetic energy emitting device; and
returning the reconditioned fluid back to the container.

16. The method of claim 15, wherein the electromagnetic energy emitting device includes an ultraviolet energy emitting device.

17. The method of claim 16, wherein the ultraviolet energy emitting device includes a number of ultraviolet lamps attached to an inner surface of the fluid reconditioning module.

18. The method of claim 15, wherein the contacting step includes providing the fluid at a flow rate of 150 to 450 gallons per minute.

19. The method of claim 15, wherein the contacting step includes providing the fluid including phosphate.

20. The method of claim 15, further comprising transporting the fluid through first and second conduits between the container and the fluid reconditioning module.
